(19) United States
(12) Patent Application Publication
(10) Pub. No.: US 2023/0146521 A1
(43) Pub. Date: May 11, 2023

(54) HEALTH CARE RESOURCES MANAGEMENT

(71) Applicants: OTTAWA HEART INSTITUTE RESEARCH CORPORATION, Ottawa (CA); INSTITUTE FOR CLINICAL EVALUATIVE SCIENCES, Toronto (CA)

(72) Inventor: Louise Sun, Ottawa (CA)

(21) Appl. No.: 17/912,887

(22) PCT Filed: Jul. 23, 2021
(86) PCT No.: PCT/CA2021/051033

§ 371 Date: Sep. 20, 2022

Related U.S. Application Data
(60) Provisional application No. 63/055,620, filed on Jul. 23, 2020.

(51) Int. Cl. G16H 40/20

(52) U.S. Cl. CPC G16H 40/20

(57) ABSTRACT

Systems and methods for managing health related resources. A length of stay (LOS) module and a waitlist module receive patient data from a database and, based on at least this data, determine probabilities for one or more patients. For the LOS module, the probability of staying for less than 2 days or more than 7 days after a specific type of surgical procedure is determined. For the waitlist module, the probability of the patient dying or becoming hospitalized within a specific amount of time while on a waiting list is determined. These probabilities are then used by a resource management module to adjust or reallocate health related resources such as critical care slots or surgical procedure scheduling.

### TECHNICAL FIELD

The present invention relates to health care facility management. More specifically, the present invention relates to systems and methods for managing surgical assets and services as well as a ward or unit of a health care facility such as a hospital.

### BACKGROUND

Healthcare resource management is an ongoing challenge for publicly funded healthcare systems because available resources are finite. This challenge has become global since the onset of the coronavirus disease (COVID-19) pandemic, as many non-emergent procedures have been postponed to preserve system capacity for patients with COVID-19. The recent (and ongoing) COVID-19 global health crisis has caused elective surgeries to be postponed to limit infectious exposure and to preserve hospital capacity. However, the ramp down in cardiac surgery volumes may result in unintended harm to patients who are at high risk of mortality if their conditions are left untreated.

Since having been declared an International Public Health Emergency by the World Health Organization (WHO) on Jan. 30, 2020, the 2019 novel coronavirus (COVID-19) outbreak has rapidly redefined societal norms and challenged healthcare systems across the globe. COVID-19 was declared as a pandemic on Mar. 11, 2020. By then, the availability of intensive care unit (ICU) resources had already begun to fall short of the increasing number of critically ill patients in some regions. Amidst this crisis, surgical patients continue to require lifesaving ICU resources. Although elective surgical procedures have been universally postponed, a significant number of patients with advanced, symptomatic cardiac diseases continue to require cardiac surgery on an urgent basis to prevent disease decompensation and death. This need challenges system capacity, given the complex comorbidities that often co-exist with cardiac surgical disease, as well as the demand for ICU monitoring after cardiac and major noncardiac surgery.

The current paradigm of triage decision-making is primarily driven by clinicians' judgment and experience, which has been shown to be highly inaccurate in predicting prolonged cardiac surgical ICU (CSICU) length of stay (LOS). Although several objective clinical CSICU LOS models have been proposed, they are all built upon small single-center datasets, lack multicenter external validation, and rely on intra- and postoperative data to achieve modest discrimination. With a goal to save more lives while maintaining an efficient and adaptable allocation of critical care resources, there is a need for better methods for managing such scarce resources such as ICU capacity.

Any suitable management system for ICU and other scarce resources (such as surgical assets) should take into account the waitlists for current patients as well as those patients coming into the health care pipeline. To date, most studies of waitlist mortality have been centered on major noncardiac surgery and/or cardiac transplantation. A study by an Alberta based group that investigated 101 cardiac waitlist deaths found that adherence to Canadian Cardiovascular Society (CCS) waitlist recommendations poorly predicted cardiac surgical waitlist mortality (c-statistic 0.577) and many patients died within recommend waitlist timeframes. The poor ability of the CCS waitlist recommendations to prevent deaths suggests a need to re-evaluate cardiac surgery triage criteria using evidence generated by Ontario data.

Accordingly, there is a need for systems and methods for managing scarce health care resources. Preferably, such systems and methods should address waitlists, mortality rates of those on the waitlists, and the length of stay in critical wards for patients. Even more preferably, such systems and methods should be simple to use and provide acceptable levels of accuracy.

### SUMMARY

The present invention provides systems and methods for managing health related resources. A length of stay (LOS) module and a waitlist module receive patient data from a database and, based on at least this data, determine probabilities for one or more patients. For the LOS module, the probability of staying for less than 2 days or more than 7 days after a specific type of surgical procedure is determined. For the waitlist module, the probability of the patient dying or becoming unexpectedly hospitalized within a specific amount of time while on a waiting list is determined. These probabilities are then used by a resource management module to adjust or reallocate health related resources used in critical care slot management, surgical procedure scheduling, or surgical waitlist management.

In a first aspect, the present invention provides a system for managing health related resources, the system comprising:
a database storing patient data;
a length of stay (LOS) module for calculating probabilities relating to a patient's projected length of stay at a health care facility;
a waitlist module for calculating probabilities relating to at least one of: a mortality and an unplanned hospitalization of at least one patient on a waiting list for health related resources;
a resource management module receiving probability outputs of said LOS module and of said waitlist module, said resource management module adjusting allocation of health related resources based on said probability outputs of said LOS module and of said waitlist module;

wherein
said probabilities calculated by said LOS module are based on patient data stored in said database;
said probabilities calculated by said waitlist module are based on patient data stored in said database.

In a second aspect, the present invention provides a system for managing health related resources, the system comprising:
a database storing patient data;
a length of stay (LOS) module for calculating probabilities relating to a patient's projected length of stay at a health care facility;
a resource management module receiving probability outputs of said LOS module, said resource management module adjusting allocation of health related resources based on said probability outputs of said LOS module;

wherein
said probabilities calculated by said LOS module are based on patient data stored in said database.

In a third aspect, the present invention provides a system for managing health related resources, the system comprising:
a database storing patient data;
a waitlist module for calculating probabilities relating to at least one of: mortality and an unplanned hospitalization of at least one patient on a waiting list for health related resources;
a resource management module receiving probability outputs of said waitlist module, said resource management module adjusting allocation of health related resources based on said probability outputs of said waitlist module;

wherein
said probabilities calculated by said waitlist module are based on patient data stored in said database.

### BRIEF DESCRIPTION OF THE DRAWINGS

The embodiments of the present invention will now be described by reference to the following figures, in which identical reference numerals in different figures indicate identical elements and in which:

FIG. 1 is a block diagram illustrating a system according to one aspect of the present invention;

FIG. 2 is a block diagram illustrating a variant of the system illustrated in FIG. 1;

FIG. 3 is a block diagram of another variant of the system illustrated in FIG. 1;

FIG. 4 is a screenshot of an application that uses the various modules of the present invention;

FIG. 5 is a screenshot of a data input screen that shows the application can simultaneously ingest data for multiple patients;

FIG. 6 is a screenshot of a data entry screen for scheduling a patient's surgery; and

FIG. 7 is a screenshot of a data entry screen for entry of data for a specific patient.

### DETAILED DESCRIPTION

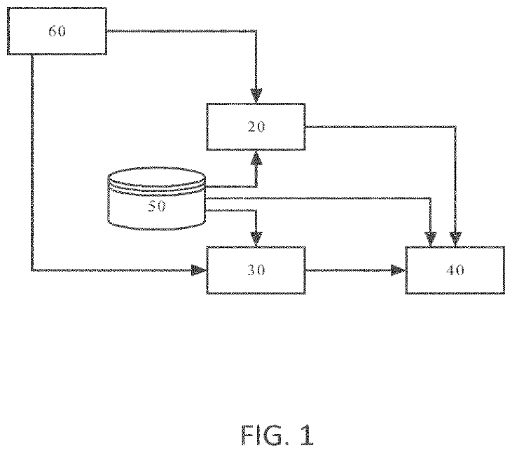

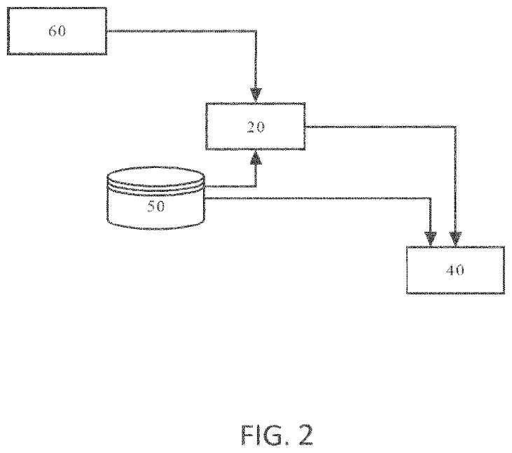

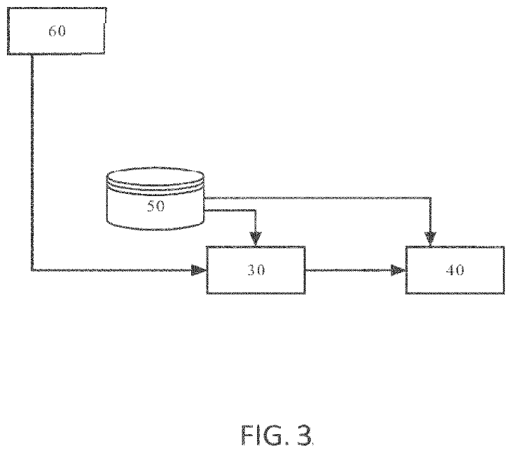

Referring to FIG. 1, a block diagram of a system according to one aspect of the invention is illustrated. As can be seen, the system 10 includes a waitlist module 20 and a LOS (length of stay) module 30. Also included is a resource management module 40. The waitlist module 20 and the LOS module 30 both receive data from a database 50 and, optionally, from a data source 60. A variant of the system 10 is illustrated in FIG. 2 and FIG. 3. In FIG. 2, only the waitlist module 20 is present while in FIG. 3, only the LOS module 30 is present. In FIG. 1, both of these modules 20, 30 are present.

In operation, the system in FIG. 1 receives data from the database 50 and, based on that data, determines probabilities relating to patients and/or resources. The waitlist module 20 determines the probability of mortality and/or hospitalization for patients in the waiting list for critical resources over a specific time window based on the available data for these patients. The LOS module 30 determines, based on available data, the probability that patients will need to consume a first lesser amount of a resource while also determining the probability that the patients will need to consume a second greater amount of that same resource. In one implementation, the resource is a length of stay in a critical care unit (or in a ward) at a health facility and the probabilities determined are whether the patient will need less than 2 days in the critical care unit or more than 7 days in the critical care unit. Other implementations may provide the exact predicted length of stay in days.

It should be clear that the system retrieves the relevant patient data from the database to determine the above noted probabilities. However, other data may also be retrieved/received from a data source such as data entry from health care professionals (e.g. an attending physician).

Once the relevant probabilities have been assessed, the resource management module 40 uses these probabilities to adjust resource allocation plans accordingly. As an example, if a patient in the waiting list has a high probability of mortality within 2 days, the system may reallocate resources to address that high probability of mortality. Similarly, the system may use the calculated probabilities for future planning. As an example, if incoming patients A and B both have an 80% chance of requiring 2 days or less in critical care, while incoming patient C has a 75% chance of requiring more than 7 days of critical care and there are currently 7 free spots available in critical care, then the system can specify that, for the next 2 days there will only be 4 spots in critical care. Similarly, the system can forecast that, from the data above and the probabilities calculated from the data, 3 days from now, there will be 6 available spots in critical care.

Other critical care resources may also be managed by the resource management module 40 using the system noted above. As another example, the scheduling of surgical procedures may be affected by the calculated probabilities of mortality for patients on the waiting list. Patient A may have a 40% probability of mortality within the next 3 days while patient B may only have a 10% probability of mortality within the next 3 days. Once a surgical slot opens up, the system may thus schedule patient A's procedure before patient B's procedure and, depending on the implementation, may assign the first available surgical team or seek out and assign the best surgical team for the procedure.

Waitlist

The waitlist module uses models created using a large sample data set. From the data set's data, suitable models were derived and, based on a number of factors, the probability of mortality for patients with specific ailments and conditions was calculated. In one implementation, the waitlist module was designed specifically to address cardiac patient waitlists. For this implementation, a cohort study of adult patients ≥18 years of age, who were placed on the waitlist for coronary artery bypass grafting (CABG), and/or aortic, mitral, tricuspid valve, or thoracic aortic surgery in Ontario within a specified date window was performed. Excluded were patients who are waitlisted for transcatheter procedures, as well as for cardiac transplantation and ventricular assist devices. As data sources for this study and model extraction, the clinical registry data from the province of Ontario, and population level administrative healthcare databases with information on all Ontario residents was used. Using unique confidential identifiers, the Ontario registry (waitlist management, date and type of procedure, physiologic and comorbidity data) was linked with the Canadian national database for hospital admissions, the Ontario physician service claims database, and the vital statistics database. These databases have been validated for many outcomes, exposures, and comorbidities. For this specific model extraction, outcomes were recorded as occurring between referral date and surgery. The primary outcome is death. The secondary outcome is non-elective hospitalization due to cardiac and all-causes. It should be clear that, in one implementation, the models allow for a user adjustable time frame in which the patient's probability of death or non-elective hospitalization is calculated. It should be clear from the description below that other models with other outcomes (such as a composite of death and non-elective hospitalization and non-elective hospitalization alone) were also created. The discussion regarding such models follows after the discussion regarding the model where death is the primary outcome.

In addition to the above parameters for the model extraction, other potential covariates were used, including (but not limited to) age, sex, smoking, hypertension, left ventricular ejection fraction (LVEF), myocardial infarction (MI) within 30 days prior to surgery, CCS angina class, New York Heart Association (NYHA) functional status, atrial fibrillation, heart failure (HF), stroke, endocarditis, peripheral arterial disease, chronic obstructive pulmonary disease, glomerular filtration rate, dialysis dependence, diabetes, anemia, redo sternotomy, type of surgery, and procedure urgency. Additionally, the following anatomic variables were evaluated: number and location of diseased coronary arteries, presence of left main, left main (LM) equivalent and proximal left anterior descending artery (LAD) disease, and the type and severity of valvular lesions. The values for these and other variables may be retrieved by the module from the database for the specific patient being assessed or the values may be retrieved/received from the data source (e.g. an attending physician or some other health professional may enter the values for the variables).

For this derivation where the primary outcome is death and the secondary outcome is non-elective hospitalization, the cohort was split into a derivation and a validation set by random selection such that ⅔ of the cohort was used to derive the model. The prediction of death was accomplished using a Cox proportional hazards model, while the prediction of non-elective hospitalization using a cause-specific hazard model within a competing risk framework. Variables were included in each of these models if their univariate P-values were <0.25, and retained if they were significant at P<0.05 in the backward elimination model or were deemed a priori to be clinically important. Scores were assigned to each retained covariate based on the method described by the Framingham group. Model calibration was assessed in the validation sample by stratifying patients into risk score strata (using thresholds based on deciles of the risk score determined in the derivation sample) and estimating the incidence of events in each risk stratum. These stratum-specific estimates of risk were compared with mean model-based estimates obtained from the risk score. This risk score was validated using the remaining randomly selected ⅓ of the cohort.

The waitlist models were based on population-based data in Ontario, the most populous and ethnically diverse province in Canada. As these models are to be used to guide decisions regarding the timing of surgery based on disease acuity and anticipated hospital resource needs at a system level, model development and validation were performed in a patient sample that is representative of the population that the system may serve. Together, these models provide rapid, data-driven decision support for clinicians, hospital administrators and policymakers, by addressing acuity and access to cardiac care when needed.

It should be clear that different models were developed to determine the probabilities for different outcomes. The model referred to above calculates the probabilities for death as the primary outcome and non-elective hospitalization and the composite of death and non-elective hospitalizations as secondary outcomes. In one variant, a model was developed such that the primary outcome was all-cause mortality that occurred between the date of acceptance onto the waitlist and the date of removal from the waitlist. For this variant, a hybrid approach of Random Forests for initial variable selection was used, followed by stepwise logistic regression for clinical interpretability and parsimony. A bootstrap sample of the data was thus used to build each of the classification trees. A random subset of variables was selected at each split, thereby constructing a large collection of decision trees with controlled variation. The trees were left unpruned in order to minimize bias. Every tree in the forest casts a “vote” for the best classification for a given observation, and the class receiving the most votes results in the prediction for that specific observation. The dataset was first sampled to create an in-bag partition (⅔ of derivation sample) to construct the decision tree, and a smaller out-of-bag partition (⅓ of derivation sample) was used to test the constructed tree and thereby evaluate its performance. As is known, Random Forests calculate estimates of variable importance for classification using the permutation variable importance measure. This is based on the decrease of classification accuracy when values of a variable in a node of a tree are permuted randomly. This model variant was based on 500 classification trees and 6 variables available for splitting at each tree node.

For this variant of the model, a subset of the top 30 predictor variables were identified out of the 40 candidate variables and these were incorporated into a logistic model. Predictor variables were entered into a multivariable backward stepwise logistic regression model based on both clinical and statistical significance, with P<0.10 for entry and P<0.05 for retention. The final prediction model was created and its results can be referred to as a Waitlist Mortality Score. The final model of this variant consisted of 11 variables. These variables included sex, type of surgery, LM-equivalent anatomy, and CCS classification and these variables were forced into the model on the basis of clinical significance. Other multivariable predictors of waitlist mortality were age, LVEF, history of HF, atrial fibrillation, dialysis, psychosis, and operative priority.

In another variant of the present invention, a different model/a variant of the models above was developed where the primary outcome was the composite of death or unplanned cardiac hospitalization, as defined by non-elective admission for heart failure, myocardial infarction, unstable angina or endocarditis between the date of acceptance and date of removal from the waitlist. For this variant, the cohort was split into a derivation and validation dataset by random selection such that ⅔ of the cohort was used to derive the model. Death or unplanned cardiac hospitalization was predicted using a Cox proportion hazard model. Predictor variables were selected using a backward stepwise algorithm with a significance threshold of P<0.1 for entry and P<0.05 for retention in the model. For continuous variables, their association with the composite outcome was examined using cubic spline analyses with five knots at percentiles 5, 27.5, 50, 72.5 and 95. As there was no violation of the linearity assumption for any of these variables, these were entered into the model as continuous values. This variant model was validated on the remaining ⅓ of the cohort.

For this variant, the predictive model consisted of 16 variables: BMI, acceptance to the waitlist during an inpatient encounter, urban residence, teaching hospital, recent MI within 30 days, CCS and NYHA classification, history of heart failure, atrial fibrillation, diabetes, glomerular filtration rate, proximal LAD disease, aortic stenosis, endocarditis, operative priority at the time of waitlisting, and type of planned surgery.

In terms of implementation, the multiple variants of the different models allow for the waitlist module to calculate different probabilities. The waitlist module can calculate probabilities for: a) mortality alone, b) hospitalization alone, or c) mortality or hospitalization. For mortality alone, two different formulas may be used—the first formula calculates the probability of death as a binary event, irrespective of length of time on the waitlist. The second formula produces time-dependent probabilities of death. As an example, when using the second formula, the probabilities of death at 15, 30, 60 and 90 days after being placed on the waitlist can be calculated. When calculating the probabilities for mortality or hospitalization or hospitalization alone, the waitlist module can be configured to calculate the time-dependent risks for specific time periods. As an example, the waitlist module can calculate the time-dependent probabilities for 15, 30, 60, and 90 days after being placed on the waitlist.

For greater clarity, Tables 4 and 5 are provided below. Table 4 details the baseline characteristics in those who died or had unplanned cardiac hospitalizations and those who did not. Table 5 details the multivariable predictors of death or unplanned cardiac hospitalization while on the waitlist. Note that the data in Tables 4 and 5 relate to cardiac patients.

In one implementation, the waitlist module can be used to cooperate with an operating room scheduling process by way of the resource management module. Such a process would be useful for optimizing the efficiency of surgical operations and for enhancing patient safety while waiting for surgery. Specifically, the predicted waitlist morbidity and mortality may be integrated with input of administrative information (from the database) at the beginning of each week (e.g., type and number of procedures anticipated, daily availability of surgeon, anesthesiologists, assistants, perfusionists, nurses) to make daily operating room (OR) schedules that will automatically take into account patient disease acuity and minimize OR cancellations, especially since such cancellations occur frequently and result in inefficient resource use as well as undue delays in lifesaving procedures. In addition, the predicted waitlist morbidity and mortality can be integrated with administrative information so that surgical teams with the most appropriate expertise are properly scheduled for relevant procedures. Such integration between the process and the waitlist module functionalities will also allow for real-time patient status updates and, in one implementation, is used to automatically rearrange the OR schedule to ensure that patients who are acutely deteriorating will receive their surgeries more urgently. The system can be configured such that triaging and OR teams receive push notifications with each scheduling change.

In one variant of the system described above, any changes, optimizations, or edits to schedules made by the system are sent to a human for validation/confirmation. Thus, any scheduling decisions made by the system are first reviewed/validated by a human before being finalized. Such a human reviewer can, when necessary, override the scheduling decisions made by the system. In the event of such an override, the system may need to rework the schedule to take into account the human override. The reworked schedule will, of course, require human approval and verification before being finalized and implemented. As noted above, the scheduling and resource management may include OR scheduling, surgical team scheduling, nurse/care worker scheduling, surgical procedure scheduling, relevant work assignments, as well as other management functions that can take into account patient care/condition.

Multiple waitlist models may be used in the waitlist module and may be used/configured depending on the desired outcome/functionality of the module. For some implementations, patients are ranked in terms of risk and those classified as high-risk (in terms of mortality or hospitalization) are given precedence/scheduled first for surgical procedures/surgical resource scheduling. Patients classified as having lower risks can be scheduled based on resource optimization methods (e.g. scheduling based on having the optimal surgical team available for higher risk/higher surgical expertise requirements and/or scheduling based on current/projected ICU (intensive care unit) capacity).

It should be clear that, depending on implementation, different pieces of data may be requested as input to the waitlist module. The different possible inputs may include: age, sex, height, weight, the type of hospital the patient is in (teaching hospital, etc.), whether the patient was waitlisted during an inpatient encounter, whether the patient has a rural residence, the CCS classification, whether the patient has had a myocardial infarction within the last 30 days, the New York Heart Association classification for the patient, whether the patient has a history of heart failure, patient conditions and characteristics such as diabetes, proximal LAD, aortic stenosis, LVEF, hypertension, atrial fibrillation, endocarditis, stroke, peripheral arterial disease, anemia, and creatinine readings. As well, the system may request other data such as the preoperative cardiogenic shock (or readings that may indicate such), the surgery type the patient requires, and the operative priority for the patient. Any subset of the above may form the input to the waitlist module. As well, other pieces of data may still be requested by the waitlist module depending on implementation.

Length of Stay

For the LOS module, two different submodules 30A and 30B were created. Each submodule used a model that predicted whether a given patient is likely to spend a given amount of time in a critical care unit. In one implementation, one submodule determined the probability that a patient would need less than 2 days of care in an intensive care unit while the other submodule determined the probability that the same patient would need more than seven days of care in the intensive care unit. For one implementation, the models derived were for cardiac patients as explained below.

In one implementation, clinical models were built to predict the likelihood of short (≤2 days) and prolonged ICU LOS (≥7 days) in patients ≥18 years of age was derived and performed. These patients were those who underwent coronary artery bypass grafting and/or aortic, mitral, and tricuspid value surgery in Ontario, Canada. Multivariable logistic regression with backward variable selection was used, along with clinical judgment, in the modeling process. For the model that predicted a short ICU stay (≤2 days), the c-statistic was 0.78 in the derivation cohort and 0.71 in the validation cohort. For the model that predicted a prolonged stay (≥7 days), the c-statistic was 0.85 in the derivation and 0.78 in the validation cohort. The models demonstrated a high degree of accuracy (tested accuracy being greater than 90%) during prospective testing.

For this implementation, an ambispective study was performed, models were derived to predict low and high ICU resource use after cardiac surgery (defined by CSICU LOS of ≤2 and ≥7 days, respectively), using data available at the University of Ottawa Heart Institute (UOHI). These models were validated using a concurrent multicenter cohort of non-UOHI cardiac surgery patients in Ontario. These models were then tested prospectively at the UOHI.

Inclusion criteria were adult patients ≥18 years of age, who underwent coronary artery bypass grafting (CABG), and/or aortic, mitral, and tricuspid valve surgery. Excluded were patients who underwent procedures requiring circulatory arrest, as well as cardiac transplantation and ventricular assist devices (VAD). For patients with multiple cardiac procedures during the study period, only the index procedure was included in the analyses.

Derivation Cohort

All 6,625 patients who underwent cardiac surgery at the UOHI within a specific date window and met the selection criteria were included in the derivation cohort. Also used were prospectively collected clinical data from a multimodular data repository that captures detailed demographics, comorbidities, procedural details and outcomes of all patients who underwent cardiac surgical procedures at the UOHI, a university-affiliated tertiary referral center that performs the full scope of cardiac operations.

Validation Cohort

The validation cohort consisted of cardiac surgical patients from 7 other cardiac care centers in Ontario, who met the selection criteria within a given date window. Also used was the clinical registry data from the province of Ontario, and population level administrative healthcare databases. The clinical registry data from the province of Ontario maintains a detailed prospective registry of all patients who undergo invasive cardiac procedures in Ontario, including demographic, comorbidity, and procedural-related information.

Using unique confidential identifiers, the clinical Ontario registry (that stored the date and type of cardiac procedures, physiologic, and comorbidity data) was linked with the Canadian database for comorbidities and hospital admissions, the provincial database for physician service claims, and the database for vital statistics. These administrative databases have been validated for many outcomes, exposures, and comorbidities, including heart failure, chronic obstructive pulmonary disease, asthma, hypertension, myocardial infarction and diabetes.

Potential covariates considered in the analyses are detailed in Table 1 and included age, sex, body mass index (BMI), smoking, hypertension, left ventricular ejection fraction (LVEF), myocardial infarction within 30 days prior to surgery, Canadian Cardiovascular Society (CCS) angina class, New York Heart Association (NYHA) class, atrial fibrillation, endocarditis, stroke, peripheral arterial disease (PAD), glomerular filtration rate (GFR), dialysis, diabetes treated with oral hypoglycemics and/or insulin, anemia, emergent operative status, preoperative cardiogenic shock, redo sternotomy and type of surgery. The definitions for these variables are provided in Supplemental Table 1 below.

Height and weight were identified from the clinical registry and procedural urgency was ascertained from the clinical registry and database for physician service claims using an established algorithm. In addition, comorbidities were identified from the clinical registry and supplemented with data from the Canadian database for comorbidities and hospital admissions and the provincial database for physician service claims using International Classification of Diseases 10th Revision (ICD-10-CA) codes within five years prior to the index procedure, according to validated algorithms. It should be clear that values for the above noted variables as well as for variables identified below may be retrieved from the database or may be received/retrieved from the data source (e.g. entered by a physician or other health care professional).

For this implementation, continuous variables were compared with a 2-sample t-test or with a Wilcoxon rank sum test for non-normally distributed data. Categorical variables were compared with a chi-square test.

In the derivation set, separate logistic regression models were developed to predict the probabilities of CSICU LOS of ≤2 days and ≥7 days, respectively. For each model, univariate logistic regression was used to examine the association of potential predictors that were available at the time of triage and were routinely reported to the clinical registry, with CSICU LOS. According to methods described by others, potential predictors of LOS with univariate P-values of <0.25 were considered for entry into a multivariable logistic regression model based on both clinical and statistical significance. A backward variable selection algorithm was used, retaining in the final multivariable model covariates with P-values of <0.05, as well as those deemed to be clinically important. The final LOS prediction models were used in the submodules of the system.

Model discrimination in both the derivation and validation datasets was assessed using the c-statistic. Calibration was assessed using the Hosmer-Lemeshow chi-square statistic and by comparing the number of observed vs. expected events in each risk quintile. Model performance was assessed using the Brier score. For each of the LOS models, a predictiveness curve was constructed in the validation dataset by plotting ordered risk percentile on the x-axis, and the probabilities of LOS≤2 days and ≥7 days, respectively, on the y-axis. Other measures of model performance, such as sensitivity, specificity, positive and negative predictive values (PPV, NPV), were determined by examining LOS in higher or lower risk groups at the optimal cutoff value.

These predictive models were tested and descriptive statistics for the testing period are presented below. Analyses were performed using SAS version 9.4 (SAS Institute, Cary, N.C.), with statistical significance defined by a two-sided P-value of <0.05.

Among the 6,625 patients in the derivation cohort, 4,201 (63.4%) stayed in the CSICU for ≤2 days and 692 (10.4%) for ≥7 days. Among 65,410 patients in the validation cohort, 50,442 (77.1%) stayed in the CSICU for ≤2 days and 3,364 (5.1%) for ≥7 days. The baseline characteristics of both cohorts were similar, with the exception that patients in the derivation cohort were younger, more likely to undergo complex surgery, to smoke, have atrial fibrillation and anemia. Patients in the validation cohort were more likely to have CCS class 4 symptoms and undergo isolated CABG (Table 1).

The multivariable predictors of short and prolonged CSICU LOS are presented in Table 2. Of the candidate covariates evaluated, younger age, female sex, lower BMI, CCS and NYHA class, higher LVEF, and the absence of atrial fibrillation, endocarditis, stroke, PAD, anemia, higher GFR, emergent operative status, preoperative cardiogenic shock, redo sternotomy, and procedure type, were predictors of short CSICU LOS.

Age and sex were forced into the prolonged LOS model on the basis of clinical significance. Other multivariable predictors of prolonged CSICU LOS were BMI, NYHA class, LVEF, hypertension, atrial fibrillation, endocarditis, anemia, GFR, emergent operative status, preoperative cardiogenic shock, redo sternotomy and procedure type.

For the short stay model, in the derivation dataset, the c-statistic of the multivariable model was 0.78 and the Hosmer-Lemeshow chi-square statistic was 12.71 (P=0.12). In the validation dataset, the c-statistic of the multivariable model was 0.71 and the Hosmer-Lemeshow chi-square statistic was 626.9 (P<0.001). The Brier score was 0.16.

Table 3A shows the observed rates of short CSICU LOS according to each risk quintile. The observed and predicted numbers of patients having LOS≤2 days were similar across all except the lowest probability quintile, where the model tended to underestimate (observed rate 53.4%, predicted 44.3%). On examining a predictiveness curve based on the data, 60% of patients had predicted probabilities exceeding the average rate of short stay. The optimal cutoff point on the ROC curve was at a predicted probability of 76.3%, with the following characteristics: sensitivity, 69.8%; specificity, 60.8%; PPV, 85.7%; NPV, 37.4%.

For the long stay model, in the derivation dataset, the c-statistic of the multivariable model was 0.85 and the Hosmer-Lemeshow chi-square statistic was 18.54 (P=0.02). In the validation dataset, the c-statistic of the multivariable model was 0.78 and the Hosmer-Lemeshow chi-square statistic was 131.43 (P<0.001). The Brier score was 0.047.

Table 3B shows a calibration table showing the rates of prolonged CSICU LOS according to each risk quintile. The number of observed cases having LOS≥7 days was similar to that predicted across all quintiles. Specifically, the average observed probability of short stay was 0.8% in quintile 1 (predicted probability 0.9%), 1.7% in quintile 2 (predicted 1.6%), 3.0% in quintile 3 (predicted 2.5%), 5.5% in quintile 4 (predicted 4.6%), and 14.8% in quintile 5 (predicted probability 17.2%). On examining a based on this data, 22% of patients had predicted probabilities that exceeded the average rate of prolonged stay. The optimal cutoff point on the ROC curve was at a predicted risk of 3.9% (sensitivity, 73.2%; specificity, 68.8%; PPV, 11.3%; NPV, 97.9%). At the 25th, 50th, and 75th percentiles of risk, sensitivities were 95.6%, 85.3%, and 64.1%, respectively, whereas negative predictive values were 99.1%, 98.5%, and 97.5%, respectively.

During a beta testing period for the two LOS models, a total of 42 patients who were evaluated with the models proceeded to have surgery on an urgent basis. Using a predictive threshold of ≥70%, 35 of 38 (92.1%) patients who were predicted to have CSICU LOS of ≤2 days actually did. One patient was predicted to have a LOS of ≥7 days but suffered intraoperative death. The remaining three patients were classified as “indeterminate” (i.e., had predicted probabilities of ≤50% for both short and prolonged LOS). Of these patients, two had a LOS of between 2-7 days and one ≥7 days.

The two models for LOS and the submodules implementing these models may be used to help optimize daily operative planning, whereby scheduling of cases with varying postoperative resource requirements could be staggered to maximize the number of urgent cases performed.

The two LOS models may be used to support triaging decisions by complementing the physician's assessment of disease acuity and clinical factors with real-world data. The potential impact of the system depends on the average CSICU LOS durations specific to each institution. At institutions with lower CSICU LOS after cardiac surgery, the system may help to identify the high resource users while, at institutions with longer CSICU LOS, the system may identify those who are likely to have a rapid transition through the CSICU. Given its robust performance in prospective validation, the two LOS models could be used to benchmark the predicted vs. observed CSICU LOS as a quality metric. They could also be used to identify patients who may benefit most from preoperative optimization (i.e., those who are mostly to require prolonged LOS).

It should also be clear that the system's resource management module may use the LOS module to predict ICU capacity needs in greater detail. Specifically, Poisson regression models may be used to predict the actual ICU LOS as a continuous variable (e.g., 4.5 days, instead of having a binary cutoff at 2 or 7 days). In the system, this predicted LOS can be integrated with administrative information from the database (such as the total ICU bed capacity, number of ICU beds available at the beginning of each week, weekly physician, housestaff and nursing availability, and type and number of procedures booked on a weekly basis) to provide daily and weekly projections of % ICU bed occupancy and number and type of staffing needed to optimize occupancy. In one variant, a model can be created to predict total hospital LOS that encompasses ICU and ward. Such a model can, in conjunction with the resource management module, be used for general hospital ward/ICU management. It should be clear that, even though the above description discusses two different LOS modules (one for a short stay and one for a longer stay), a single LOS module may be used. Such a module may, depending on the implementation, predict the actual total hospital LOS as a continuous variable, or determine the probability that a patient would have a minimum length of hospitalization. Conversely, such a module may determine the probability that a patient would have a length of stay that is a maximum.

The system may be used as part of an overall application used to provide ICU capacity projections and to make staffing recommendations to optimize capacity in an automated fashion. In addition, while the above description is made with respect to ICU or critical care spots/beds and the scheduling of surgical procedures, the system and its components may be used for the management of other scarce medical resources. This may include the management and dispensing of medications, medicaments, physician/caregiver time, allocation of consultation hours for physicians and/or specialists, and other health related resources. The various embodiments of the various systems according to the present invention may be part of a larger system used in scheduling, capacity planning, and overall management of scare hospital/health care resources. As such, while the above may refer to the LOS and the waitlist modules as being together in one system, each module may be deployed by itself in separate systems.

It should be clear that, while the above descriptions specify cardiac patients as being the subjects for model derivation, models for non-cardiac patients are also possible. The procedure for deriving models for non-cardiac patients would be the same as for cardiac patients but would, of course, involve data for non-cardiac patients. Accordingly, the advantages of the various aspects of the present invention can be extended to include non-cardiac patients.

In terms of implementation, the system may be implemented on a server from which the various modules are operating. The integrated output of the resource management module may be accessed by users on any number of data processing devices including desktops, laptops, mobile devices, and smartphones. The system may also be integrated into a larger management system that operates/manages a health care facility such as a hospital.

It should also be clear that while the above discusses a system that includes both the LOS module and the waitlist module, systems that only include one of the two modules are possible. For such an implementation, only the LOS module or only the waitlist module would be present and, other than that, the system would operate as above. For such a system, whichever module is present, the resource management module would be configured to receive the present module's probability output and use that output to manage the scarce medical or health resources. Of course, if the LOS module is not present, then the resource management module would be unable to forecast the critical care or ICU slots/beds based on the LOS predictions. Similarly, the resource management module would be unable to rearrange surgical procedures based on a projected mortality or unplanned hospitalization risk of patients on the waiting list if the waitlist module is not present.

In other implementations, the waitlist module and the LOS module may both be implemented as standalone applications that execute/operate either online or on conventional computing devices. Alternatively, the various modules of the present invention may be implemented as part of an electronic health record system or as part of a larger system used in or with a health related facility. As an example, the waitlist module may be resident on a mobile device or may be accessed as an online resource for use by health care professionals as necessary. Similarly, the LOS module may be a standalone online or cloud based resource that is accessed by health care professionals as needed. For these examples, the values for the variables necessary to calculate the relevant probabilities may be entered by one or more health care professionals. The resulting probabilities would then be provided to these professionals as standalone numbers for use by the professionals as necessary.

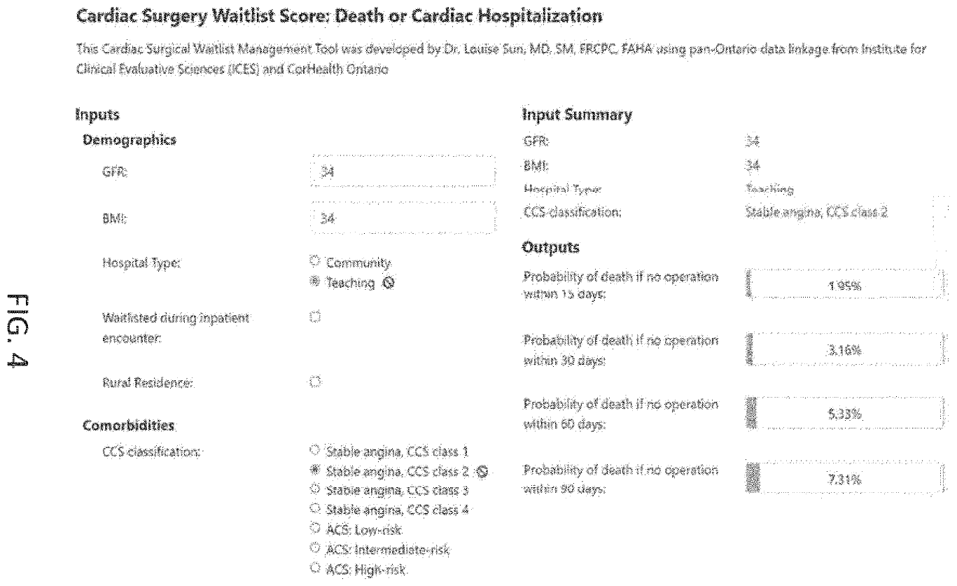

Referring to FIG. 4, a screenshot of an application that uses the modules of the present invention is illustrated. The inputs to the application can be seen and these inputs are used to calculate the probabilities relating to one or more specific patients.

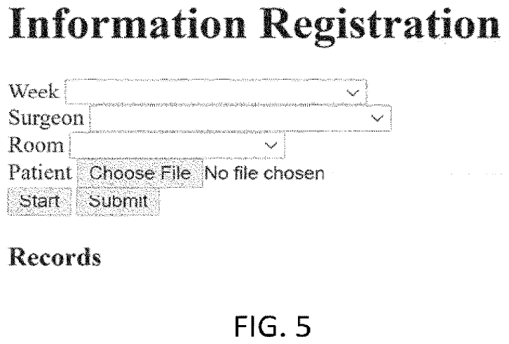

Referring to FIG. 5, another screenshot of data input to the application that uses the above modules is illustrated. For this implementation, the system can simultaneously ingest data from multiple patients (by way of a single data file) and can use this data to calculate the probabilities for each patient and to optimally schedule scarce resources based on these probabilities.

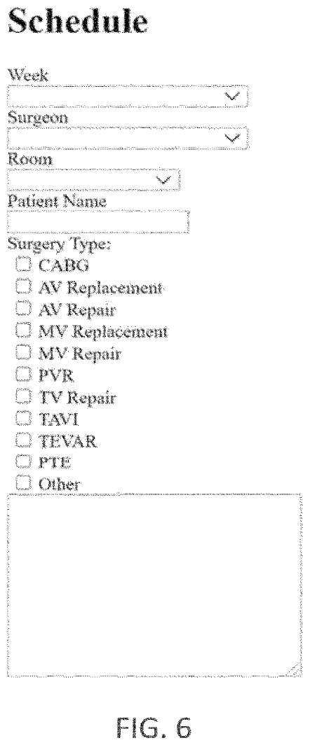

As part of a scheduling application, FIG. 6 is a screenshot of data necessary to schedule an individual patient for surgery. As can be seen, the desired week, surgeon, room, and patient is entered along with the type of surgery. Based on these inputs, the application can optimally schedule the surgery based on the probabilities calculated for this patient and other patients who are similarly waiting for surgery. Similar screens may be used to schedule other scarce hospital resources as necessary.

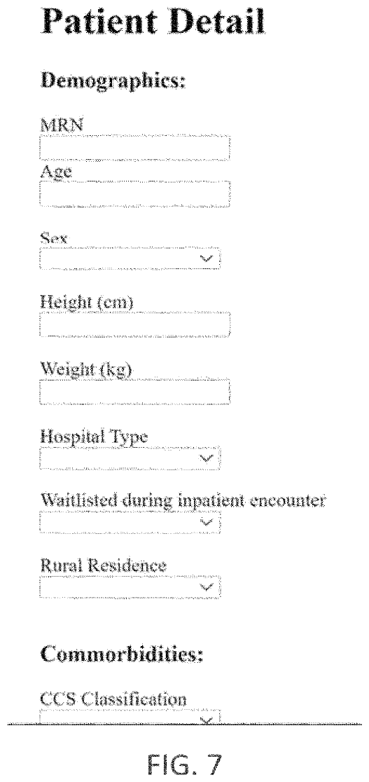

Referring to FIG. 7, a portion of a data entry screen is illustrated for the entry of data for a specific patient. The data entered may be used in the calculation of the probabilities as noted above. The various fields for this data entry screen are detailed above.

The tables referred to above are provided below.

TABLE 1
Baseline characteristics of the derivation and validation cohorts

| | Derivation | | Validation | |
|---|---|---|---|---|
| Variable | (n = 6,625) | | (n = 79,196) | |
| Demographic | | | | |
| Age, median (IQR), year | 59 | (67-75) | 67 | (60-75) |
| Age, n (%), year | | | | |
| ≤40 | 188 | (2.8) | 850 | (1.3) |
| 41-64 | 2.596 | (39.2) | 25.315 | (38.7) |
| 65-74 | 2.163 | (32.7) | 22.690 | (34.7) |
| 75-84 | 1.507 | (22.8) | 15.993 | (24.5) |
| ≥85 | 171 | (2.6) | 1.629 | (2.5%) |
| Female sex, n (%) | 1.851 | (27.9) | 15.993 | (24.5%) |
| Body mass index, n (%), kg m² | | | | |
| <18.0 | 61 | (0.9) | 0 | |
| 18.1-24.9 | 1.779 | (26.9) | 17.059 | (26.1) |
| 25.0-29.9 | 2.577 | (38.9) | 25.769 | (39.4) |
| 30.0-34.9 | 1.446 | (21.8) | 14.896 | (22.3) |
| ≥35.0 | 762 | (11.5) | 7.686 | (11.8) |
| Comorbidities, n (%) | | | | |
| Hypertension | 4.855 | (73.3) | 56.521 | (86.4) |
| Myocardial infarction within | 1.407 | (21.2) | 16.185 | (24.7) |
| 30 days of surgery | | | | |
| Canadian Cardiovascular | | | | |
| Society classification | | | | |
| 0 | 2.751 | (41.5) | 12.620 | (19.3) |
| 1 | 492 | (7.4) | 5.583 | (8.5) |
| 2 | 1.070 | (16.2) | 10.574 | (16.2) |
| 3 | 1.100 | (16.6) | 10.963 | (16.8) |
| 4 | 1.212 | (18.3) | 25.670 | (39.2) |
| New York Heart | | | | |
| Association classification | | | | |
| 0 | 2.497 | (37.7) | 17.365 | (26.5) |
| 1 | 765 | (11.6) | 28.849 | (44.1) |
| 2 | 1.430 | (21.6) | 8.839 | (13.5) |
| 3 | 1.526 | (23.0) | 8.386 | (12.8) |
| 4 | 407 | (6.1) | 1.971 | (3.0) |
| Left ventricular ejection fraction | | | | |
| ≥50% | 4.914 | (74.2) | 44.844 | (68.6) |
| 35-49% | 1.009 | (15.2) | 14.228 | (21.8) |
| 20-35% | 474 | (7.2) | 5.421 | (8.3) |
| <20% | 228 | (3.4) | 917 | (1.4) |
| Atrial fibrillation | 1.117 | (16.9) | 4.704 | (7.2) |
| Endocarditis | 128 | (1.9) | 847 | (1.3) |
| Smoker (active or former) | 4.186 | (63.2) | 11.726 | (17.9) |
| Stroke | 748 | (11.3) | 6.759 | (10.3) |
| Peripheral arterial disease | 718 | (10.8) | 8.220 | (12.6) |
| Diabetes on medications | 1.761 | (26.6) | 20.652 | (31.6) |
| Anemia | 2.244 | (33.9) | 6.912 | (10.6) |
| Glomerular filtration rate, | | | | |
| mL min per 1.73 m² | | | | |
| ≥60 | 4.921 | (74.3) | 49.260 | (75.3) |
| 30-59 | 1.486 | (22.4) | 13.995 | (21.4) |
| <30 | 218 | (3.3) | 2.155 | (3.3) |
| Dialysis | 102 | (1.5) | 1.432 | (2.2) |
| Operative characteristics, n (%) | | | | |
| Emergent procedure | 531 | (8.0) | 9.930 | (15.2) |
| Preoperative cardiogenic shock | 244 | (3.7) | 2.700 | (4.1) |
| Redo sternotomy | 539 | (8.1) | 2.110 | (3.2) |
| Type of Surgery | | | | |
| CABG | 2.908 | (43.9) | 47.136 | (72.1) |
| Single valve | 1.176 | (17.8) | 9.245 | (14.1) |
| Valve(s) = CABG | 2.541 | (38.4) | 9.029 | (13.8) |

IQR = interquartile range;
CABG = coronary artery bypass grafting.

SUPPLEMENTARY TABLE 1
Covariates and their definitions.
These definitions are in keeping with definitions
employed by EuroSCORE¹ and or the STS database.²

| Covariates | Definition |
|---|---|
| Hypertension | A. BP >140 mmHg systolic or >90 mmHg diastolic in |
| | patients without diabetes or chronic kidney disease: |
| | or |
| | B. BP >130 mmHg systolic or >30 mmHg diastolic on at |
| | least two occasions in patients with diabetes or |
| | chronic kidney disease; |
| | C. History of hypertension treated with medication, |
| | diet, and/or exercise |
| Atrial | Documented history of paroxysmal or permanent atrial |
| fibrillation | fibrillation |
| Endocarditis | Endocarditis that is currently being treated with |
| | antibiotics |
| Peripheral | A. Claudication either with exertion or at rest: |
| arterial | B. Amputation for arterial vascular insufficiency: |
| disease | C. Vascular reconstruction, bypass surgery, or |
| | percutaneous intervention to the extremities: |
| | documented abdominal aneurysm with or without repair: |
| | D. Positive noninvasive test (ankle brachial index |
| | 0.9, ultrasound, MRA, CTA of >50% in any peripheral |
| | artery) or angiographic imaging |
| Diabetes on | Diabetes mellitus treated with oral hypoglycemic |
| medications | and/or insulin |
| Anemia | Defined by the World Health Organization³ (<130 g/L |
| | for men and <120 g/L for women), based on the |
| | hemoglobin concentration measured closest to the |
| | time of surgery. |
| Glomerular | Calculated using the Cockcroft-Gault formula⁴ |
| filtration rate | |
| Emergent | Surgery that must take place within 24 hours of |
| surgery | acute hospital admission |
| Preoperative | Requirement for inotropic support with evidence of |
| cardiogenic | end organ hypoperfusion or dysfunction or intraaortic |
| shock | balloon pump in situ before surgery |

References:
¹EuroSCORE. European System for Cardiac Operative Risk Evaluation. Available from URL: http://www.euroscore.org.
²The Society of Thoracic Surgeons National Database. Available from URL: http://www.euroscore.org
³Organization WH. Nutritional Anaemias: Report of a WHO Scientific Group. Geneva, Switzerland: World Health Organization. 1968.
⁴Cockcroft D W, Gault M H. Prediction of creatinine clearance from serum creatinine. Nephron. 1976: 16(1): 31-41.

TABLE 2
Multivariate analysis of patients with cardiac surgical intensive
care unit length of stay of ≤2 days vs. >2 days.

| | Model | | Wald | |
|---|---|---|---|---|
| Variable | β-Coefficient | OR (95% CI) | Chi-Square | P Value |
| Demographic | | | | |
| Age, year | | | | |
| ≤40 | NA | Reference | Reference | NA |
| 41-64 | −0.192 | 0.83 (0.56-1.23) | 0.92 | 0.339 |
| 65-74 | −0.404 | 0.67 (0.45-1.00) | 3.95 | 0.047 |
| 75-84 | −0.515 | 0.60 (0.40-0.90) | 6.09 | 0.014 |
| ≥85 | −0.795 | 0.46 (0.27-0.77) | 8.99 | 0.003 |
| Female sex | −0.169 | 0.84 (0.74-0.97) | 6.18 | 0.013 |
| Body mass index, kg m² | | | | |
| <18.0 | −0.0408 | 0.96 (0.54-1.72) | 0.019 | 0.891 |
| 18.0-24.9 | NA | Reference | Reference | NA |
| 25.0-29.9 | −0.194 | 0.82 (0.71-0.96) | 6.12 | 0.013 |
| 30.0-34.9 | −0.461 | 0.63 (0.53-0.75) | 26.09 | <0.0001 |
| ≥35.0 | −0.703 | 0.50 (0.40-0.61) | 43.05 | <0.0001 |
| Comorbidities | | | | |
| CCS classification | | | | |
| 0 | NA | Reference | Reference | NA |
| 1 | −0.0087 | 0.99 (0.78-1.26) | 0.0051 | 0.94 |
| 2 | 0.147 | 1.16 (0.96-1.41) | 2.25 | 0.13 |
| 3 | 0.0341 | 1.04 (0.86-1.25) | 0.12 | 0.73 |
| 4 | −0.197 | 0.82 (0.67-1.00) | 3.72 | 0.05 |
| NYHA classification | | | | |
| 0 | NA | Reference | Reference | NA |
| 1 | −0.0656 | 0.94 (0.76-1.16) | 0.38 | 0.54 |
| 2 | −0.208 | 0.81 (0.69-0.96) | 5.94 | 0.01 |
| 3 | −0.538 | 0.59 (0.50-0.69) | 40.11 | <0.0001 |
| 4 | −1.288 | 0.28 (0.20-0.38) | 60.57 | <0.0001 |
| Left ventricular | | | | |
| ejection fraction | | | | |
| ≥50% | NA | Reference | Reference | NA |
| 35-49% | −0.386 | 0.68 (0.68-0.80) | 21.69 | <0.0001 |
| 20-35% | −1.043 | 0.35 (0.28-0.44) | 80.81 | <0.0001 |
| <20% | −1.479 | 0.23 (0.16-0.34) | 57.81 | <0.0001 |
| Atrial fibrillation | −0.302 | 0.74 (0.63-0.87) | 14.25 | 0.0002 |
| Endocarditis | −0.660 | 0.52 (0.33-0.81) | 8.66 | 0.003 |
| Stroke | −0.250 | 0.78 (0.65-0.93) | 7.40 | 0.007 |
| Peripheral arterial | −0.194 | 0.82 (0.69-0.99) | 4.28 | 0.04 |
| disease | | | | |
| Anemia | −0.373 | 0.69 (0.61-0.79) | 31.65 | <0.0001 |
| GFR, mL min 1.73 m² | | | | |
| ≥60 | NA | Reference | Reference | NA |
| 30-59 | −0.463 | 0.63 (0.54-0.74) | 32.45 | <0.0001 |
| <30 | −0.805 | 0.45 (0.32-0.63) | 21.72 | <0.0001 |
| Operative | | | | |
| characteristics | | | | |
| Emergent procedure | −0.914 | 0.40 (0.31-0.52) | 48.40 | <0.0001 |
| Preoperative | −1.218 | 0.30 (0.18-0.48) | 24.59 | <0.0001 |
| cardiogenic shock | | | | |
| Redo sternotomy | −0.539 | 0.58 (0.47-0.72) | 25.27 | <0.0001 |
| Type of Surgery | | | | |
| CABG | NA | Reference | Reference | NA |
| Single valve | 0.0131 | 1.01 (0.82-1.25) | 0.015 | 0.90 |
| Valve(s) = CABG | −0.785 | 0.46 (0.39-0.54) | 88.88 | <0.0001 |

OR = odds ratio;
CI = confidence interval;
MI = myocardial infarction;
CCS = Canadian Cardiovascular Society;
NYHA = New York Heart Association;
GFR = glomerular filtration rate;
CABG = coronary artery bypass grafting.

TABLE 3a
Observed versus predicted number of patients with a cardiac surgical intensive
care unit length of stay of ≤2 days in the validation cohort. The 95% confidence
intervals were obtained through 200 bootstraps with replacement.

| | Observed | | Predicted | | |
|---|---|---|---|---|---|
| Risk Quintile | Number | Rate (95% CI) | Number | Rate (95% CI) | OR (95% CI) |
| 1 (Low likelihood) | 6988 | 0.53 (0.52-0.54) | 5792.6 | 0.44 (0.44-0.45) | Reference |
| 2 (Low-moderate) | 9672 | 0.74 (0.73-0.75) | 9379.3 | 0.72 (0.72-0.72) | 2.47 (2.35-2.61) |
| 3 (Moderate) | 10614 | 0.81 (0.80-0.82) | 10659.1 | 0.82 (0.81-0.82) | 3.75 (3.55-3.97) |
| 4 (Moderate-high) | 11356 | 0.87 (0.86-0.87) | 11437.9 | 0.87 (0.87-0.87) | 5 58 (5.25-2.93) |
| 5 (High) | 11812 | 0.91 (0.90-0.91) | 11903.7 | 0.91 (0.91-0.91) | 8.44 (7.88-9.04) |

TABLE 3b
Observed versus predicted number of patients with a cardiac surgical intensive
care unit length of stay of ≥7 days in the validation cohort. The 95% confidence
intervals were obtained through 200 bootstraps with replacement.

| | Observed | | | Predicted | | | | |
|---|---|---|---|---|---|---|---|---|
| Risk Quintile | Number | Rate (95% CT) | | Number | Rate (95% CI) | | OR (95% CI) | |
| 1 (Low likelihood) | 111 | 0.008 | (0.007-0.01) | 128.5 | 0.009 | (0.009-0.009) | Reference | |
| 2 (Low-moderate) | 207 | 0.017 | (0.014-0.019) | 194.9 | 0.016 | (0.016-0.016) | 2.06 | (1.63-2.60) |
| 3 (Moderate) | 400 | 0.030 | (0.027-0.033) | 330.2 | 0.025 | (0.025-0.025) | 3.83 | (3.10-4.73) |
| 4 (Moderate-high) | 710 | 0.055 | (0.050-0.058) | 594.4 | 0.046 | (0.045-0.046) | 7.08 | (5.79-8.66) |
| 5 (High) | 1936 | 0.15 | (0.14-0.15) | 2253.2 | 0.17 | (0.17-0.18) | 21.26 | (17.53-25.78) |

TABLE 4
Baseline characteristics in those who died or had unplanned
cardiac hospitalizations and those who did not

| | No event | | Event | | Standardized |
|---|---|---|---|---|---|
| Variable | N = 59,342 | | N = 3,033 | | Differences |
| Demographics | | | | | |
| Age, Mean ± SD, y | 66.3 | (10.9) | 67.1 | (10.3) | 0.07 |
| Age, Median (IQR), y | 67 | (60-74) | 68 | (60-74) | 0.05 |
| Female sex, No. (%) | 15,626 | (26.3%) | 756 | (24.9%) | 0.03 |
| BMI, Mean ± SD, kg/m² | 28.95 | (5.56) | 28.56 | (5.38) | 0.07 |
| BMI, Median (IQR), kg/m² | 28 | (25-32) | 28 | (25-31) | 0.07 |
| Rural residence, No. (%) | 50,619 | (85.3%) | 2,619 | (86.4%) | 0.03 |
| Hospital type, No. (%) | | | | | |
| Community | 14,953 | (25.2%) | 379 | (12.5%) | 0.33 |
| Teaching | 44,389 | (74.8%) | 2,654 | (87.5%) | |
| Waitlisted during | 2,913 | (4.9%) | 770 | (25.4%) | 0.6 |
| inpatient encounter, | | | | | |
| No. (%) | | | | | |
| Comorbidities | | | | | |
| Hypertension, No. (%) | 49,488 | (83.4%) | 2,662 | (87.8%) | 0.12 |
| Atrial fibrillation, | 7,212 | (12.2%) | 418 | (13.8%) | 0.05 |
| No. (%) | | | | | |
| Recent MI, No. (%) | 2,119 | (3.6%) | 437 | (14.4%) | 0.39 |
| CCS classification, | | | | | |
| No. (%) | | | | | |
| 0 | 21,386 | (36.0%) | 575 | (19.0%) | 0.39 |
| 1 | 8,805 | (14.8%) | 483 | (15.9%) | 0.03 |
| 2 | 14,989 | (25.3%) | 569 | (18.8%) | 0.16 |
| 3 | 11,828 | (19.9%) | 603 | (19.9%) | 0 |
| 4 | 1,112 | (1.9%) | 163 | (5.4%) | 0.19 |
| Low-risk ACS | 858 | (1.4%) | 360 | (11.9%) | 0.43 |
| Intermediate-risk ACS | 339 | (0.6%) | 260 | (8.6%) | 0.39 |
| High-risk ACS | 25 | (0.0%) | 20 | (0.7%) | 0.1 |
| LM or LM equivalent | 18,319 | (30.9%) | 1,311 | (43.2%) | 0.26 |
| disease, No. (%) | | | | | |
| Proximal LAD disease, | 19,571 | (33.0%) | 1,346 | (44.4%) | 0.24 |
| No. (%) | | | | | |
| Previous PCI, No. (%) | 6,047 | (10.2%) | 375 | (12.4%) | 0.07 |
| Left ventricular | | | | | |
| ejection fraction, No. (%) | | | | | |
| ≥50% | 46,417 | (78.2%) | 2,108 | (69.5%) | 0.2 |
| 35-49% | 9,367 | (15.8%) | 611 | (20.1%) | 0.11 |
| 20-35% | 3,057 | (5.2%) | 273 | (9.0%) | 0.15 |
| <20% | 501 | (0.8%) | 41 | (1.4%) | 0.05 |
| NYHA classification, | | | | | |
| No. (%) | | | | | |
| 1 | 35,438 | (59.7%) | 2,038 | (67.2%) | 0.16 |
| 2 | 12,920 | (21.8%) | 409 | (13.5%) | 0.22 |
| 3 | 10,251 | (17.3%) | 460 | (15.2%) | 0.06 |
| 4 | 733 | (1.2%) | 126 | (4.2%) | 0.18 |
| Heart failure, No. (%) | 13,843 | (23.3%) | 968 | (31.9%) | 0.19 |
| Moderate-severe mitral | 6,951 | (11.7%) | 220 | (7.3%) | 0.15 |
| regurgitation, No. (%) | | | | | |
| Moderate-severe aortic | 2,278 | (3.8%) | 69 | (2.3%) | 0.09 |
| regurgitation, No. (%) | | | | | |
| Severe aortic stenosis, | 18,980 | (32.0%) | 687 | (22.7%) | 0.21 |
| No. (%) | | | | | |
| Endocarditis, No. (%) | | | | | |
| None | 58,852 | (99.2%) | 3,007 | (99.1%) | 0 |
| Acute | 154 | (0.3%) | 13 | (0.4%) | 0.03 |
| Subacute | 336 | (0.6%) | 13 | (0.4%) | 0.02 |
| Cerebrovascular disease, | 5,451 | (9.2%) | 314 | (10.4%) | 0.04 |
| No. (%) | | | | | |
| Peripheral arterial | 7,942 | (13.4%) | 412 | (13.6%) | 0.01 |
| disease, No. (%) | | | | | |
| Smoking status, No. (%) | | | | | |
| Never | 29,074 | (49.0%) | 1,448 | (47.7%) | 0.03 |
| Current | 9,129 | (15.4%) | 604 | (19.9%) | 0.12 |
| Former | 21,139 | (35.6%) | 981 | (32.3%) | 0.07 |
| COPD, No. (%) | 13,111 | (22.1%) | 795 | (26.2%) | 0.1 |
| Diabetes, No. (%) | 23,204 | (39.1%) | 1,435 | (47.3%) | 0.17 |
| Dyslipidemia, No. (%) | 39,600 | (66.7%) | 2,116 | (69.8%) | 0.07 |
| GFR, Mean ± SD, | 86.2 | (34.1) | 82.0 | (35.2) | 0.12 |
| mL/min/1.73 m² | | | | | |
| GFR, Median (IQR), | 82 | (62-105) | 79 | (58-103) | 0.11 |
| mL/min/1.73 m² | | | | | |
| Dialysis, No. (%) | 1,074 | (1.8%) | 90 | (3.0%) | 0.08 |
| Anemia, No. (%) | 2,344 | (3.9%) | 197 | (6.5%) | 0.11 |
| Liver disease, No. (%) | 561 | (0.9%) | 37 | (1.2%) | 0.03 |
| Alcohol abuse, No. (%) | 509 | (0.9%) | 43 | (1.4%) | 0.05 |
| Dementia, No. (%) | 656 | (1.1%) | 47 | (1.5%) | 0.04 |
| Depression, No. (%) | 415 | (0.7%) | 51 | (1.7%) | 0.09 |
| Psychosis, No. (%) | 70 | (0.1%) | 6 | (0.2%) | 0.02 |
| Primary cancer, No. (%) | 2,887 | (4.9%) | 150 | (4.9%) | 0 |
| Metastatic cancer, No. (%) | 287 | (0.5%) | 18 | (0.6%) | 0.02 |
| Operative characteristics | | | | | |
| Surgery type, No. (%) | | | | | |
| CABG | 30,481 | (51.4%) | 2,091 | (68.9%) | 0.36 |
| Valve | 18,781 | (31.6%) | 500 | (16.5%) | 0.36 |
| CABG + Valve | 7,518 | (12.7%) | 415 | (13.7%) | 0.03 |
| Thoracic Aorta | 2,562 | (4.3%) | 27 | (0.9%) | 0.22 |
| Redo-Sternotomy, No. (%) | 2,047 | (3.4%) | 131 | (4.3%) | 0.05 |
| Cardiogenic Shock, No. (%) | *52-59 | | *1-5 | | 0.01 |
| Operative priority, | | | | | |
| No. (%) | | | | | |
| Urgent | 20,296 | (34.2%) | 988 | (32.6%) | 0.03 |
| Semi-urgent | 11,180 | (18.8%) | 844 | (27.8%) | 0.21 |
| Elective | 27,866 | (47.0%) | 1,201 | (39.6%) | 0.15 |
| Recommend maximum wait | 43.7 | (34.2) | 41.3 | (31.0) | 0.07 |
| time, Mean ± SD, d | | | | | |
| Recommend maximum wait | 40 | (14-71) | 31 | (14-62) | 0.05 |
| time, Median (IQR), d | | | | | |
| Adherence to recommended | 31,126 | (52.5%) | 1,999 | (65.9%) | 0.28 |
| wait time**, No. (%) | | | | | |
| All-cause ED visits on | 0.1 | (0.5) | 0.3 | (0.6) | 0.28 |
| the waitlist, Mean ± SD | | | | | |
| All-cause ED visits on | 0 | (0-0) | 0 | (0-0) | 0.35 |
| the waitlist, Median (IQR) | | | | | |
| All-cause outpatient | 2.1 | (1.8) | 1.0 | (1.5) | 0.63 |
| physician visits on the | | | | | |
| waitlist, Mean ± SD | | | | | |
| All-cause outpatient | 2 | (1-3) | 1 | (0-2) | 0.75 |
| physician visits on the | | | | | |
| waitlist, Median (IQR) | | | | | |

*Data suppressed due to small cells
**Adherence is defined as adhering to procedure-specific wait times recommended by the Canadian Cardiovascular Society Access to Care Working Group (1).
Abbreviations:
SD = standard deviation;
IQR = interquartile range;
BMI = body mass index;
MI = myocardial infarction;
CCS = Canadian Cardiovascular Society;
ACS = acute coronary syndrome;
LM = left main;
LAD = left anterior descending;
PCI = percutaneous coronary intervention;
LVEF = left ventricular ejection fraction;
NYHA = New York Heart Association;
COPD = chronic obstructive pulmonary disease;
GFR = glomerular filtration rate;
CABG = coronary artery bypass grafting;
ED = emergency department

TABLE 5
Multivariate predictors of death or unplanned
cardiac hospitalization while on the waitlist

| | Model | | | |
|---|---|---|---|---|
| Variable | β-Coefficient | HR (95% CI) | | P-value |
| Demographics | | | | |
| GFR | −0.00220 | 1 | (1-1) | 0.003 |
| BMI | −0.01390 | 0.99 | (0.98-1) | 0.004 |
| Teaching vs. | 0.58030 | 1.79 | (1.56-2.05) | <.0001 |
| community hospital | | | | |
| Waitlisted during | 1.62000 | 5.05 | (4.48-5.7) | <.0001 |
| inpatient encounter | | | | |
| Rural Residence | −0.15610 | 0.86 | (0.75-0.97) | 0.02 |
| Comorbidities | | | | |
| CCS classification | | | | |
| 0 | NA | Reference | | NA |
| 1 | 0.46610 | 1.59 | (1.36-1.87) | <.0001 |
| 2 | 0.17870 | 1.2 | (1.02-1.4) | 0.03 |
| 3 | 0.40100 | 1.49 | (1.27-1.75) | <.0001 |
| 4 | 1.00830 | 2.74 | (2.18-3.45) | <.0001 |
| Low-risk ACS | 1.79390 | 6.01 | (4.96-7.29) | <.0001 |
| Intermediate-risk ACS | 2.12500 | 8.37 | (6.68-10.49) | <.0001 |
| High-risk ACS | 2.08940 | 8.08 | (4.81-13.56) | <.0001 |
| Recent MI | −0.17960 | 0.84 | (0.72-0.97) | 0.02 |
| NYHA classification | | | | |
| 1 | NA | Reference | | NA |
| 2 | −0.32440 | 0.72 | (0.62-0.84) | <.0001 |
| 3 | −0.06690 | 0.94 | (0.8-1.09) | 0.4 |
| 4 | 0.57800 | 1.78 | (1.4-2.26) | <.0001 |
| Heart failure | 0.33430 | 1.4 | (1.25-1.56) | <.0001 |
| Atrial Fibrillation | 0.23010 | 1.26 | (1.1-1.44) | 0.0007 |
| Diabetes | 0.12470 | 1.13 | (1.03-1.24) | 0.008 |
| Proximal LAD | 0.10790 | 1.11 | (1.01-1.23) | 0.03 |
| Aortic Stenosis | 0.24900 | 1.28 | (1.08-1.52) | 0.004 |
| Endocarditis | | | | |
| None | NA | Reference | | NA |
| Acute | 0.77580 | 2.17 | (1.16-4.08) | 0.02 |
| Subacute | −0.24120 | 0.79 | (0.37-1.66) | 0.5 |
| Operative characteristics | | | | |
| Surgery type | | | | |
| CABG | NA | Reference | | NA |
| Valve | −1.09820 | 0.33 | (0.27-0.41) | <.0001 |
| CABG + Valve | −0.49070 | 0.61 | (0.49-0.76) | <.0001 |
| Thoracic Aorta | −1.81340 | 0.16 | (0.1-0.27) | <.0001 |
| Operative priority | | | | |
| Urgent | 0.37080 | 1.45 | (1.24-1.69) | <.0001 |
| Semi-urgent | 0.40440 | 1.5 | (1.34-1.68) | <.0001 |

| | | | |
|---|---|---|---|
| Elective | NA | Reference | NA |

Abbreviations:
BMI = body mass index;
CCS = Canadian Cardiovascular Society;
ACS = acute coronary syndrome;
MI = myocardial infarction;
NYHA = New York Heart Association;
LAD = left anterior descending;
GFR = glomerular filtration rate;
CABG = coronary artery bypass grafting

It should be clear that the various aspects of the present invention may be implemented as software modules in an overall software system. As such, the present invention may thus take the form of computer executable instructions that, when executed, implements various software modules with predefined functions.

The embodiments of the invention may be executed by a computer processor or similar device programmed in the manner of method steps, or may be executed by an electronic system which is provided with means for executing these steps. Similarly, an electronic memory means such as computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network. Various embodiments of the differing aspects of the invention may also take the form of computer programs that are available for use and/or download from online repositories. Similarly, other embodiments may take the form of computer software that is stored and/or executable and/or hosted from an online repository or from an online server.

Embodiments of the invention may be implemented in any conventional computer programming language. For example, preferred embodiments may be implemented in a procedural programming language (e.g., “C” or “Go”) or an object-oriented language (e.g., “C++”, “java”, “javascript”, “PHP”, “PYTHON” or “C#”). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for managing health related resources, the system comprising:
a database storing patient data;
a length of stay (LOS) module for calculating probabilities relating to a patient's projected length of stay at a health care facility;
a waitlist module for calculating probabilities relating to at least one of: a mortality and an unplanned hospitalization of at least one patient on a waiting list for health related resources;
a resource management module receiving probability outputs of said LOS module and of said waitlist module, said resource management module adjusting allocation of health related resources based on said probability outputs of said LOS module and of said waitlist module;
wherein
said probabilities calculated by said LOS module are based on patient data stored in said database;
said probabilities calculated by said waitlist module are based on patient data stored in said database.

2. The system according to claim 1, wherein said LOS module also receives additional data from a data source.

3. The system according to claim 1, wherein said waitlist module also receives additional data from a data source.

4. The system according to claim 1, wherein said LOS module comprises a submodule for calculating probabilities that a patient will require less than a specific number of days of critical care after cardiac surgery.

5. The system according to claim 1, wherein said LOS module comprises a submodule for calculating probabilities that a patient will require more than a specific number of days of critical care after cardiac surgery.

6. The system according to claim 1, wherein said patient is a cardiac patient.

7. The system according to claim 1, wherein said health related resources comprises at least one of:
ICU bed capacity;
ICU bed availability;
hospital bed capacity;
hospital bed availability;
physician time;
house staff availability;
nursing availability;
scheduling of surgical procedures;
scheduling of operating room teams;
scheduling of operating room time;
medications;
medicaments;
allocation of consultation hours for physicians; and
allocation of consultation time for specialists.

8. The system according to claim 1, wherein said system is part of an application used to provide optimized operating room scheduling.

9. The system according to claim 1, wherein said patient is a non-cardiac patient.

10. The system according to claim 1, wherein said LOS module comprises a submodule for calculating probabilities that a patient will require more than a specified number of days of critical care after cardiac surgery.

11. The system according to claim 1, wherein said LOS module comprises a submodule for calculating a probable number of days of critical care for said patient after cardiac surgery.

12. A system for managing health related resources, the system comprising:
a database storing patient data;
a length of stay (LOS) module for calculating probabilities relating to a patient's projected length of stay at a health care facility;
a resource management module receiving probability outputs of said LOS module, said resource management module adjusting allocation of health related resources based on said probability outputs of said LOS module;
wherein
said probabilities calculated by said LOS module are based on patient data stored in said database.

13. The system according to claim 12, wherein said LOS module also receives additional data from a data source.

14. The system according to claim 12, wherein said LOS module comprises a submodule for calculating probabilities that a patient will require less than a specific number of days of critical care after cardiac surgery.

15. The system according to claim 14, wherein said specific number of days of critical care after cardiac surgery is 2.

16. The system according to claim 12, wherein said LOS module comprises a submodule for calculating probabilities that a patient will require more than a specific number of days of critical care after cardiac surgery.

17. The system according to claim 16, wherein said specific number of days of critical care after cardiac surgery is 7.

18. The system according to claim 12, wherein said patient is a cardiac patient.

19. The system according to claim 12, wherein said health related resources comprises at least one of:
ICU bed capacity;
ICU bed availability;
hospital bed capacity;
hospital bed availability;
physician time;
house staff availability;
nursing availability;
scheduling of surgical procedures;
scheduling of operating room teams;
scheduling of operating room time;
medications;
medicaments;
allocation of consultation hours for physicians; and
allocation of consultation time for specialists.

20. The system according to claim 12, wherein said system is used in an application used to provide optimized operating room scheduling.

21. The system according to claim 12, wherein said patient is a non-cardiac patient.

22. The system according to claim 12, wherein said LOS module comprises a submodule for calculating probabilities that a patient will require more than a specified number of days of critical care after cardiac surgery.

23. The system according to claim 12, wherein said LOS module comprises a submodule for calculating a probable number of days of critical care for said patient after cardiac surgery.

24. A system for managing health related resources, the system comprising:
a database storing patient data;
a waitlist module for calculating probabilities relating to at least one of: a mortality and an unplanned hospitalization of at least one patient on a waiting list for health related resources;
a resource management module receiving probability outputs of said waitlist module, said resource management module adjusting allocation of health related resources based on said probability outputs of said waitlist module;
wherein
said probabilities calculated by said waitlist module are based on patient data stored in said database.

25. The system according to claim 24, wherein said patient is a cardiac patient.

26. The system according to claim 24, wherein said health related resources comprises at least one of:
ICU bed capacity;
ICU bed availability;
hospital bed capacity;
hospital bed availability;
physician time;
house staff availability;
nursing availability;
scheduling of surgical procedures;
scheduling of operating room teams;
scheduling of operating room time;
medications;
medicaments;
allocation of consultation hours for physicians; and
allocation of consultation time for specialists.

27. The system according to claim 24, wherein said system is used in an application used to provide optimized operating room scheduling.

28. The system according to claim 24, wherein said patient is a non-cardiac patient.
